(19) United States
(12) Patent Application Publication
(10) Pub. No.: US 2017/0340243 A1
(43) Pub. Date: Nov. 30, 2017

(54) Spatial Detection and Alignment of an Implantable Biosensing Platform

(71) Applicant: Biorasis, Inc., Storrs, CT (US)

(72) Inventors: Faquir Jain, Storrs, CT (US); Fotios Papadimitrakopoulos, West Hartford, CT (US); Antonio Costa, Hartford, CT (US); Michail Kastellorizios, Willington, CT (US); Allen Legassey, Storrs Mansfield, CT (US)

(21) Appl. No.: 15/457,347

(22) Filed: Mar. 13, 2017

Related U.S. Application Data
(60) Provisional application No. 62/307,443, filed on Mar. 12, 2016.

(51) Int. Cl. A61B 5/07; A61B 5/145; A61B 5/06; A61B 5/00; A61B 5/055

(52) U.S. Cl. CPC A61B 5/076; A61B 5/742; A61B 5/055; A61B 5/062; A61B 5/0084; A61B 5/14503; A61B 2560/0219; A61B 2562/0223; A61B 2562/0238

(57) ABSTRACT

A system and method is outlined for a wearable external device that communicates with a fully implantable miniaturized biosensor platform providing fast spatial detection and accurate assessment of the position and orientation of the implant within highly scattering tissue. The device and method provides spatial (x, y) position, depth (z) and rotational (φ) state of the implantable biosensor platform. The spatial (x, y) position allows the ability to turn-on only one out of an entire array of LEDs that is in line-of-sight with the implant in order to conserve power. Similarly, the depth and rotational coordinates information is used to adjust the output light intensity of the selected light emitters to compensate the power delivered to the implant. The above attributes render the system compatible for usage during intense physical activity and for added user comfort through improved skin ventilation.

### CROSS REFERENCE TO RELATED APPLICATIONS

This application is related to and claims the benefit of the filing date of U.S. Provisional Patent Application Ser. No. 62/307,443 filed Mar. 12, 2016, the contents of which are incorporated herein by reference in its entirety.

### STATEMENT REGARDING FEDERALLY SPONSORED RESEARCH

The United States Government has certain rights in this invention pursuant to U.S. Army Medical Research and Materiel Command Grant No. W81XWH-15-C-0069.

### FIELD OF THE INVENTION

The present invention relates generally to implantable biosensing platforms and more specifically to the detection and alignment of the implantable biosensing platforms.

### BACKGROUND OF THE INVENTION

Biosensing platforms, or biosensors, for medical applications have significant promises as a means to diagnose and to manage diseases. A biosensor can be any device that detects any chemical or physical change, converts that signal into an electrical or chemical signal and transmits the response to a secondary device. An implantable biosensor is a biosensor that is implantable within the body of a patient and may be implanted within the various layers of skin (e.g. intradermal), as well as within subcutaneous tissue, intramuscularly or within the vasculature. Unfortunately however, once the biosensor has been fully implanted, difficulty typically arises in determining the exact spatial location of the implant.

This is undesirable because if the location of the implant is not known, communication with the implant may be impossible or difficult and unreliable at best. One reason for this difficulty is that after the biosensor has been implanted, the skin heals and provides a barrier to visually locating the biosensor. Another reason for this difficulty is that a proximity communicator, that is typically provided and intended to interface with the implanted biosensor, might also drift from alignment. As such, the proximity communicator may also need to be re-aligned for accurate communication.

### SUMMARY OF THE INVENTION

Fully implantable biosensors require energy to function and to transmit data to/from external devices. As an example, energy may be supplied to a biosensor by a built-in battery or by harnessing electromagnetic radiation via photovoltaic cells (optical powering), or radio-frequency coils (RF powering), embedded within the biosensor. In the case of optical powering, light scattering of the surrounding tissue rapidly diminishes the incident light on the photovoltaic cell(s). As a result of this, the use of photovoltaic cells embedded in a biosensor necessitates the electromagnetic radiation to be directed towards the photovoltaic cells in order for the photovoltaic cells to produce sufficient electrical energy. This energy in turn power the electronic and optoelectronic devices located on the implanted biosensor platform. Therefore, in order for an external device to direct the electromagnetic radiation to the photovoltaic cells of the biosensor, the spatial location of the biosensor must be identified.

Once the fully implantable biosensor is implanted, there is not a tangible or direct means of communication from the biosensor to an external device. This matter is overcome by transmitting data wirelessly. Transmitted data can be of many forms; for example, electromagnetic radiation and various forms of telemetry. As a means of communication, one approach is to embed a source of electromagnetic radiation into the implantable biosensor; for example, a light emitting diode (LED) or a laser. The source of the electromagnetic radiation may operate at a specific wavelength or over a range of wavelengths. The external device containing one or more photodetectors can be used to detect the amplitude or the frequency of the emitted electromagnetic radiation. For optical communication, the emitted light from the biosensor can also be time-delayed, or operate at a different wavelength than the powering source, or be a combination of a time-delay and at a different wavelength than the powering source. As an example, the frequency may be related to the concentration of a specific analyte, e.g. glucose, lactate, molecular oxygen, glycerol, glutamate, hydrogen peroxide, etc. In order for the photodetector to detect the emitted radiation from the implanted biosensor, the implanted biosensor must be aligned with the external device such that the electromagnetic radiation source (e.g. a light emitting diode or diode array or laser diode) is in close proximity to the photodetector of the external device.

In the case of miniaturized, implantable biosensors (with dimensions of few millimeters or smaller), the strong scattering nature of skin tissue makes it challenging to identify their precise location. Moreover, in order to promote patient adoption and long-term comfort (i.e. from days to years), the external device must be loosely attached to the person's body to allow sufficient skin ventilation. The latter adds substantial design complexity since implant localization must be constantly performed (typically in milliseconds range) in order to account for active lifestyles (e.g. while running), while also maintaining robust powering and communication protocols with the implant and paired external device.

This invention describes three prime examples to readily identify the spatial (x, y), depth (z) and rotational (φ) location of a miniaturized implant within highly scattering tissue; while at the same time ensuring that both the powering light source(s) and receiving photodetector(s) on the external device are situated directly over the implant and further accounting for implant rotation. These examples ensure optimal device performance with a loosely attached external device to promote patient adoption and long-term comfort:

A) The first example uses magnetic materials (e.g. permanent magnets, electromagnets or micro/nanosized magnetic particles) localized within or around the implantable biosensor platform. Applicable substances for these magnets include ferromagnetic, ferroelectric, multiferroics, magnetoelectric and ferroelectric materials. A subcategory of these magnets is comprised of high strength magnetic materials selected from a list samarium, iron, ferrite, samarium boron garnet, etc. The spatial location of the biosensor can be determined and mapped using the following approach: the implantable biosensor partially comprises of, or it is outfitted with, magnetic material; the external device uses magnetic field detecting sensors that are capable of detecting the magnetic field generated by the polarized material located within the biosensor platform; and the external device uses signal processing algorithms to generate a two- and three-dimensional spatial location of the implanted biosensor. In the case of electromagnets, the biosensor would first need to power the electromagnet(s) prior to magnetic mapping by the external device.

B) The second example comprises of an implantable biosensor that is equipped with materials or devices that they are non-magnetic or minimally magnetic in nature, yet in the presence of an external magnetic or electromagnetic field, they interact with the field and alter it. Such materials are diamagnetic, paramagnetic, antiferromagnetic (i.e. spin glass), and other non-magnetic or magnetically polarizable substances. Subcategories of magnetically polarizable material include traditional metals (Au, Pt, Pd, Cu, Al, etc.), organic conductors and graphitic materials (such as nanotubes, graphene, etc.). The invention extends also into configuring the aforementioned materials into coils and complex 2D and 3D architectures with cores of magnetic polarizable substances to impart sufficient interaction with external magnetic fields. In this manner, the magnetically susceptible, or magnetically polarizable material(s) and devices are embedded within the implantable biosensor or surround the implantable biosensor in a form of one or more coils in either open-loop or closed-loop configurations. In this approach, the external device is appropriately modified to induce a magnetic field. The interaction of the external magnetic field with the non-magnetic or minimally magnetic materials and devices located on the implant can alter the induced magnetic field. An array of magnetic field detecting sensors (located on the external device) are then used to map the changes in the induced magnetic field and determine the spatial (x, y), depth (z) and rotational (φ) position of a miniaturized implant within a highly scattering tissue. This approach is important for elderly and/or high-risk users, who may wish to undergo magnetic resonance imaging (MRI) without the need to remove the implanted biosensor.

C) The third example employs the use of the photodetector (PD) array located on the external device (or proximity communicator) and two or more light sources (e.g. LEDs or lasers) located within the implantable biosensor that are oriented at a defined angle with each other (e.g. at 90°). By illuminating all of the light sources on the external device, one can ensure that the implanted device is powered and the two or more on-board LEDs or lasers are activated. By simultaneously scanning each photodetector in the photodetector array, the amplitude of the emitted light (i.e. intensity) can be established at each photodetector. The result is an intensity map from the photodetector array that can then be used to determine the spatial (x, y), depth (z) and rotational (φ) position of a miniaturized implant within a highly scattering tissue. This approach is also compatible with MRI test.

Radio-frequency identification (RFID) chips or tags embedded in the biosensor can also be used to assist in precise location of the implantable biosensor. This borrows similarities on the third example where one RFID tag can be embedded at the top of the sensor and a second RFID tag at the bottom. Each RFID tag could contain unique information that distinguishes the location of the tag on the device (e.g. a unique identification for the top of the biosensor and a unique identification for the bottom of the biosensor). The external device can then use either a single radio-frequency (RF) antenna or an array of RF antennas to detect the location of each RFID tag. The detection can be based on signal intensity and frequency. The detection of the RFID tags can then be used to generate a two-dimensional or three-dimensional spatial location of the implanted biosensor.

The current invention addresses two major issues associated with the subject matter. The first relates to the spatial mapping of a fully implantable biosensor. The second relates to establishing line-of-sight between the powering and communication devices along with appropriate compensation to account for implant rotation (φ) away from its optimal orientation. Moreover, it is important to stress that depending on the depth (z) and the rotation (φ) state of the implant from its optimal alignment (φ=0°), both the power and the line-of-sight must be appropriately compensated. These compensations are important since: (i) an increased implant depth (z) accounts for greater optical attenuation of the powering light; and (ii) implant rotation (φ) reduces the cross-section of the on-board photovoltaic device, hence generating less power for the implant. Consequently, the depth (z) and the rotation (φ) state of the implant play an important role in assessing the exact level of the power need for optimal function of the implant, which at the same time prolong battery lifetime for the external unit. Moreover, this invention also applies for fluorescence (excitation and photoluminescence) and Raman (excitation and back scattering) communication protocols as well. The above summary uses many examples to explain the invention, but is not exhaustive. A detailed description is provided in the sections below.

A wearable system for the spatial detection of a fully implantable miniaturized biosensor within body tissue, using minimal energy is provided, wherein the system includes an external control unit, a miniaturized, fully implantable biosensor platform, wherein the external control unit comprises an array of magnetic field detecting sensors, an array of light emitters, and an array of light photodetectors, wherein the external control unit also contains a microprocessor which interfaces with a powering source, a data acquisition module, a display, a magnetic field source, and other components, and wherein the miniaturized biosensor platform is outfitted with light powered photovoltaic cells and one or more light emitters to optically transmit detected concentration values of various analytes, and wherein the miniaturized biosensor platform comprises one or more miniaturized magnets, wherein the magnetic field of the miniaturized magnets is sensed and imaged by the magnetic field detecting sensor array in the external control unit to provide the assessment of the spatial (x, y) position, depth (z) and rotational (φ) state of the implantable biosensor platform, wherein the spatial (x, y) position allows the ability to turn on one or more light emitters in the array of the external control unit, that are in a line-of-sight alignment with the miniaturized biosensor platform, wherein the depth and rotational coordinates information is used by the microprocessor in the external control unit to adjust the output light intensity of the selected light emitters, as well as power adjacent light emitters to compensate for the rotation of the photovoltaic cells, wherein the spatial and rotational position is used by the microprocessor to turn on one or more photodetectors in the array of the external control unit that are also aligned with the miniaturized biosensor platform, wherein the changes in the spatial position and orientation of the external control unit with respect to the miniaturized biosensor platform is assessed to account for random motion caused by intense physical activity of the user.

A method for spatial detection of a miniaturized fully implantable biosensor within a body tissue is provided, wherein the method comprises magnetic alignment and minimizes energy usage via an algorithm facilitating alignment for both optical powering and optical communication units, wherein the algorithm is located in the microprocessor of an external control unit which interfaces with a miniaturized biosensor platform, wherein the algorithm interfaces with an array of magnetic field detecting sensors, an array of light emitters, and an array of light photodetectors within the said external control unit, wherein the algorithm also interfaces with powering source, data acquisition module, display, magnetic field sources, and other components within the external control unit, wherein said algorithm interfaces with the miniaturized biosensor platform through its light powered photovoltaic cells and one or more light emitters that optically transmits the detected concentration values of various analytes to the external control unit, wherein the algorithm senses the position of the miniaturized biosensor platform through the mapping of the magnetic field generated by one or more miniaturized magnets located on it, and imaged by the magnetic field detecting sensor array in the external unit to provide the precise assessment of the spatial (x, y) position, depth (z) and rotational (φ) state of the implantable biosensor platform, wherein the algorithm uses the precise spatial (x, y) position to turn on one or more light emitters in the array of the external control unit, which are aligned by line-of-sight with the miniaturized biosensor platform, wherein the algorithm uses the depth and rotational coordinates information to adjust the output light intensity of the selected light emitters, as well as power adjacent light emitters to compensate for the rotation of the photovoltaic cells wherein the algorithm uses the precise spatial and rotational position to turn on one or more photodetectors in the array of the external control unit that are also aligned with the miniaturized biosensor platform, wherein the algorithm accounts for changes in the spatial position and orientation of the external control unit with respect to the miniaturized biosensor platform to account for random motion caused by intense physical activity of the user.

A method for spatial detection of a miniaturized fully implantable biosensor within a body tissue is provided that comprises optical alignment and minimizes energy usage via an algorithm facilitating alignment for both optical powering and optical communication units, wherein the algorithm is located in the microprocessor of an external control unit which interfaces with a miniaturized biosensor platform, wherein the algorithm interfaces with an array of light emitters, and a array of light photodetectors within the external control unit, wherein the algorithm also interfaces with powering source, data acquisition module, display, and other components within the external control unit, wherein the algorithm interfaces with the miniaturized biosensor platform through its light powered photovoltaic cells and a pair of light emitters oriented at about 90° from each other and at about 45° with respect to the bottom of the external control unit, wherein the algorithm senses the position of the miniaturized biosensor platform through the mapping of the intensity generated on the array of light photodetectors to provide the precise assessment of the spatial (x, y) position, depth (z) and rotational (□) state of the implantable biosensor platform, wherein the algorithm uses the precise spatial (x, y) position to turn on one or more light emitters in the array of the external control unit, which are aligned by line-of-sight with the miniaturized biosensor platform, wherein the algorithm uses the depth and rotational coordinates information to adjust the output light intensity of the selected light emitters, as well as power adjacent light emitters to compensate for the rotation of the photovoltaic cells wherein the algorithm uses the precise spatial and rotational position to turn on one or more photodetectors in the array of the external control unit that are also aligned with the miniaturized biosensor platform, wherein the algorithm accounts for changes in the spatial position and orientation of the external control unit with respect to the miniaturized biosensor platform to account for random motion caused by intense physical activity of the user.

### BRIEF DESCRIPTION OF THE DRAWINGS

The foregoing and other features and advantages of the present invention will be more fully understood from the following detailed description of illustrative embodiments, taken in conjunction with the accompanying drawings in which like elements are numbered alike:

FIG. 1 Example of the implanted biosensor with an external device, also termed proximity communicator.

FIG. 2 Main components of the external device (proximity communicator).

FIG. 3 illustrates magnetic detecting sensors (e.g. Hall-effect sensors or giant magnetoresistance sensors) placed in an array on a platform combined with arrays of electromagnetic radiation sources (e.g. LEDs or lasers) and photodetectors on a second platform.

FIG. 4 illustrates the combination of multiple array elements on a single platform.

FIG. 5 illustrates the combination of an array of magnetic field detecting sensors with some sensors at a ninety degree angle.

FIG. 6 illustrates the combination of an array of magnetic field detecting sensors stacked in two layers at different distances above the skin layer.

FIG. 7 illustrates the combining of the magnetic detecting sensors array with the arrays of electromagnetic radiation (EMR) sources and photodetectors. Magnetic fields are illustrated as rings protruding from magnets. Light is emitted from the implanted biosensor EMR source and the proximity communicator EMR source.

FIG. 8 illustrates that the external device is capable of determining the spatial (x, y), depth (z) and rotational (φ) position of a miniaturized implant within a highly scattering tissue.

FIG. 9a illustrates magnetic interacting/polarizing materials and devices within the implanted biosensor to alter the magnetic field pattern produced by permanent magnetic field generators situated within the external device.

FIG. 9b illustrates magnetic interacting/polarizing materials and devices within the implanted biosensor to alter the magnetic field pattern produced by oscillating magnetic field generators situated within the external device

FIG. 10A illustrates configurations of magnetic interacting/polarizing materials and devices within or on the implanted biosensor a single coil wrapped around the outside of the biosensor.

FIG. 10B illustrates configurations of magnetic interacting/polarizing materials and devices within or on the implanted biosensor two coils at different sizes wrapped within the biosensor.

FIG. 10C illustrates configurations of magnetic interacting/polarizing materials and devices within or on the implanted biosensor miniature electromagnetic coils placed within the implant.

FIG. 11 utilizes the photodetector (PD) array of the external device (proximity communicator) to map the emission from two on-board LEDs or lasers situated within the implantable biosensor, which are oriented at 90° with respect to each other. Implant rotation generates different PD responses as a function of rotational angle (φ).

FIG. 12 illustrates an example method for the external device with magnetic field detecting sensors to detect, align, and communicate with the biosensor.

FIG. 13 illustrates an example method for the external device using electromagnetic radiation feedback control to detect, align, and communicate with the biosensor

### DETAILED DESCRIPTION OF THE INVENTION

The present invention relates primarily to the versatility of establishing a robust communication protocol with a fully implantable biosensor(s) and/or other fully implantable medical device(s). In one embodiment of the invention, a system and underlying method(s) to determine the exact location of a fully implantable biosensor with respect to an external frame of reference (e.g. a frame of reference with respect to a proximity communicator or a watch-like external device) is provided. Moreover, once the spatial location of the device is determined, system and underlying methods are outlined to communicate with such a device, permitting an active life style.

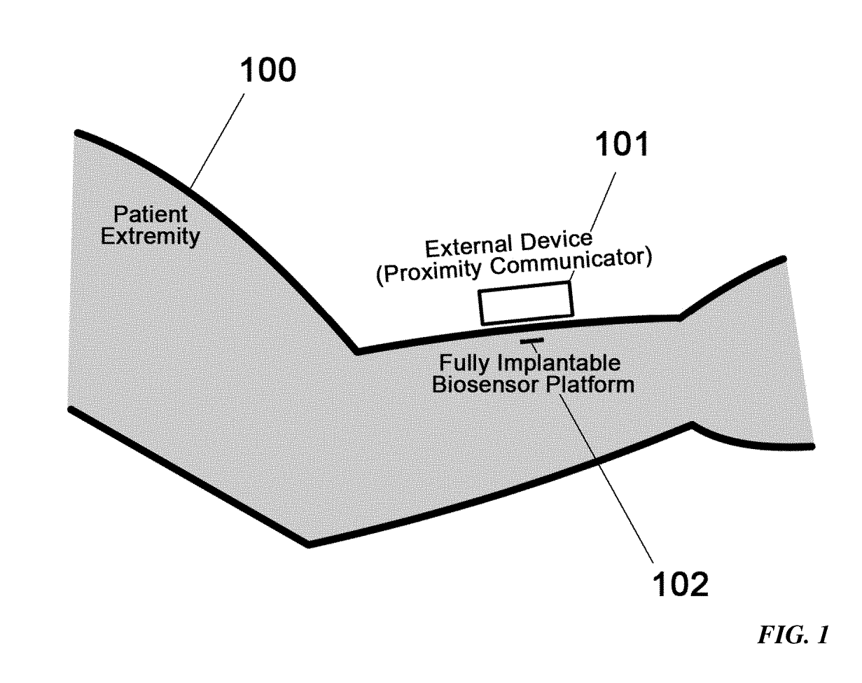

The present invention provides a device and method where the spatial location of a fully implantable biosensor can be readily accessed and at the same time a line-of-sight powering and communication can be established with an external device (proximity communicator). FIG. 1 illustrates the implantable biosensor implanted in a human's extremity 100. The device is described as an external device or “proximity communicator” 101 that comprises multiple layers of electrical components (e.g. a data acquisition module 201 and processing unit or computer 200) and circuitry 202. This device is used to detect and communicate with an implantable biosensor 102. As an example, this device can be in direct or indirect contact with an animal or human subject. The proximity communicator does not require permanent fixation to the subject.

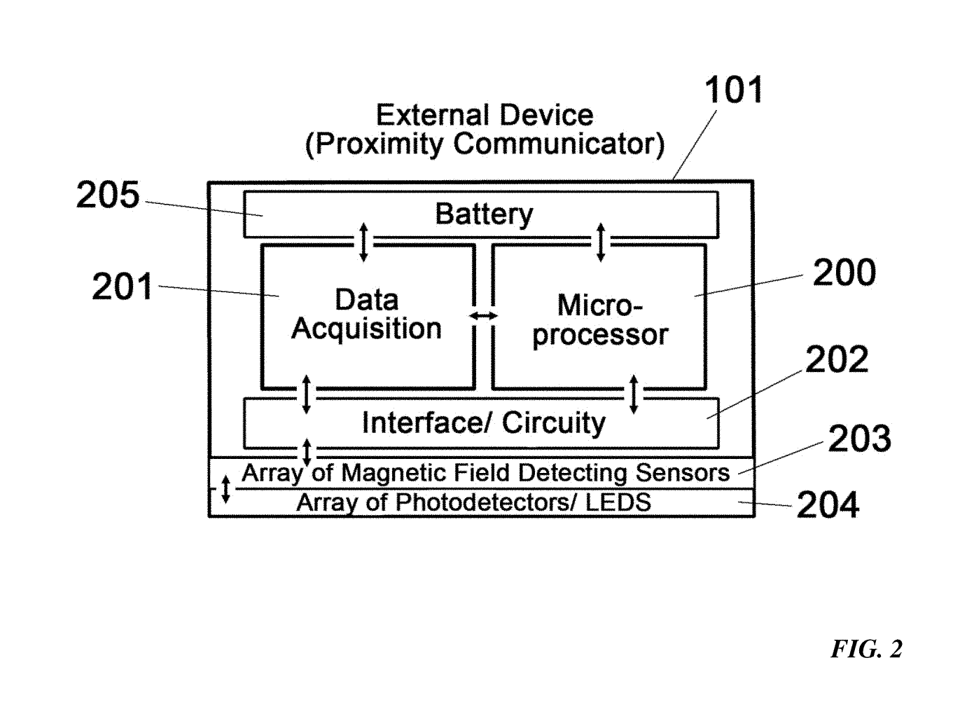

As shown in FIG. 2, the main constituents of the external device vary with respect to the three prime examples described in order to identify the spatial (x, y), depth (z) and rotational (φ) location of a miniaturized implant 102 within the highly scattering tissue underneath the skin 103. These constituents comprise of a data acquisition module 201, a micro-processor or computer 200 and associated display, array of magnetic field detecting sensors 203, array of photodetectors and light emitters 204, battery 205, external magnetic field generator 206, and interface circuitry 202, the latter of which establishes multiplexing, signal amplification and other requirements for proper function of the aforementioned arrays and devices. The external device 101 can be in direct or indirect contact with the human subject, animal, or plant and does not require permanent fixation to the subject/object (i.e. it can be loosely bound).

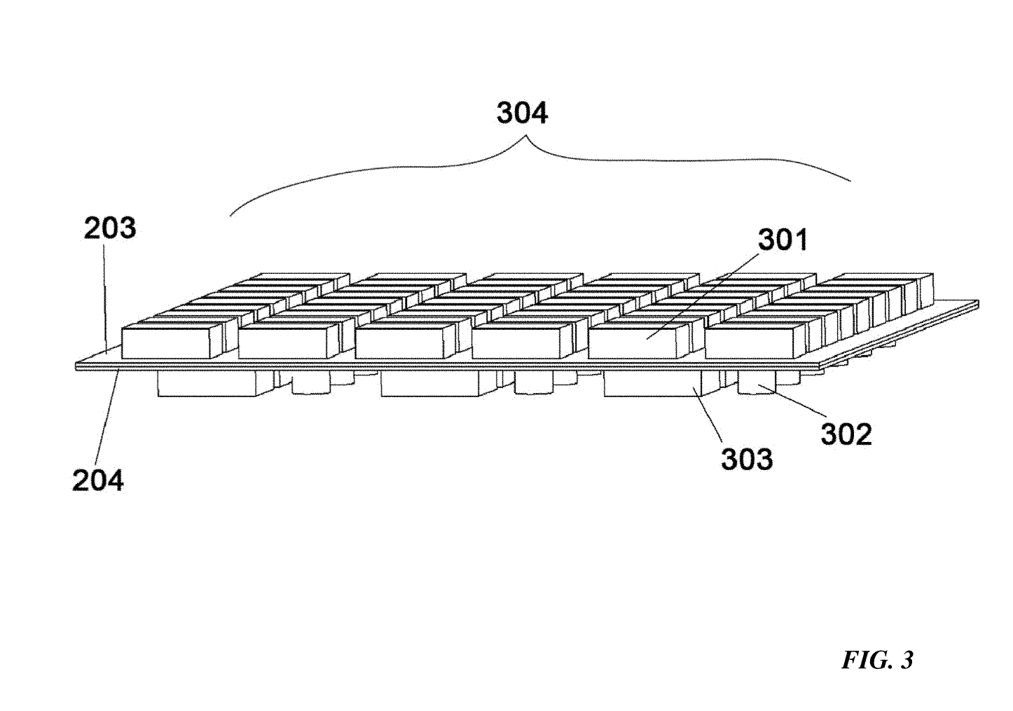

FIG. 3 shows an exemplary architecture of the two bottom layers of the external device 101. Layer 203 consists of either a single magnetic field detecting sensor 301 or an array of magnetic field detecting sensors 203 mounted on a platform such as a printed circuit board. The purpose of these sensors is to convert the presence of a magnetic field into an electrical signal such as voltage or current. An array of magnetic field detecting sensors 203 that are simultaneously converting a magnetic field into an electrical signal enables the spatial detection of any magnetic material within a particular region of interest (ROI). For example, this ROI may be a 2-inch by 2-inch area of skin. Two examples of magnetic field detecting sensors are Hall Effect sensors and giant magnetoresistance sensors (GMRs). The magnetic field detecting sensors are positioned in such a way as to detect the magnetic field. For Hall Effect sensors, the sensor element should be positioned perpendicular to the magnetic field for optimal detection.

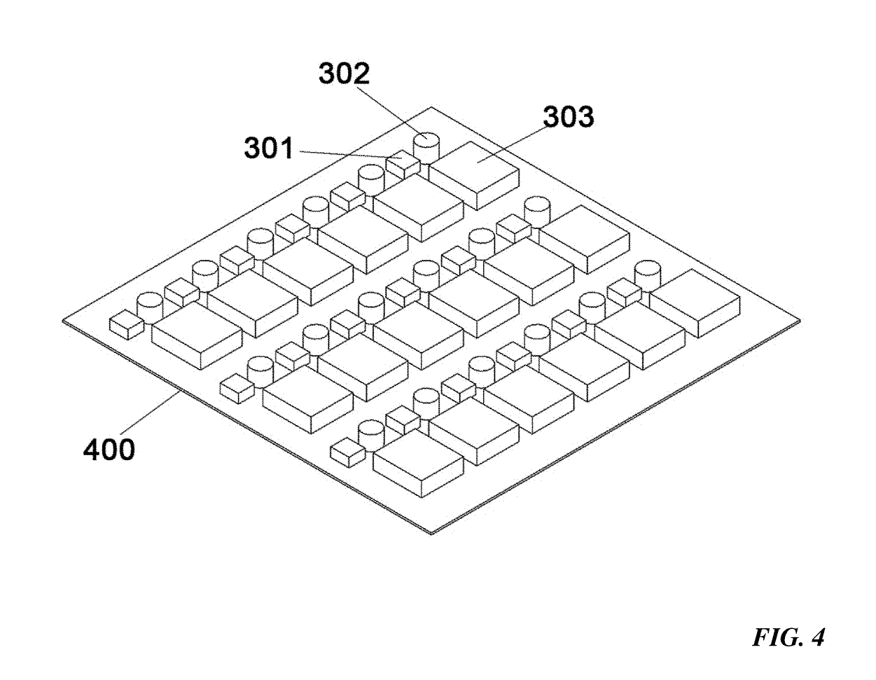

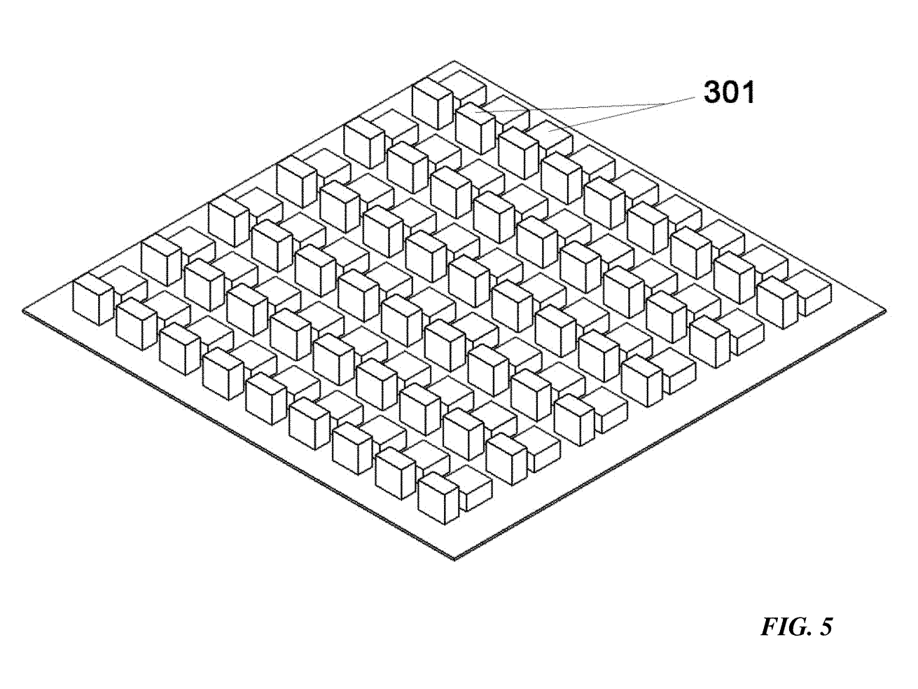

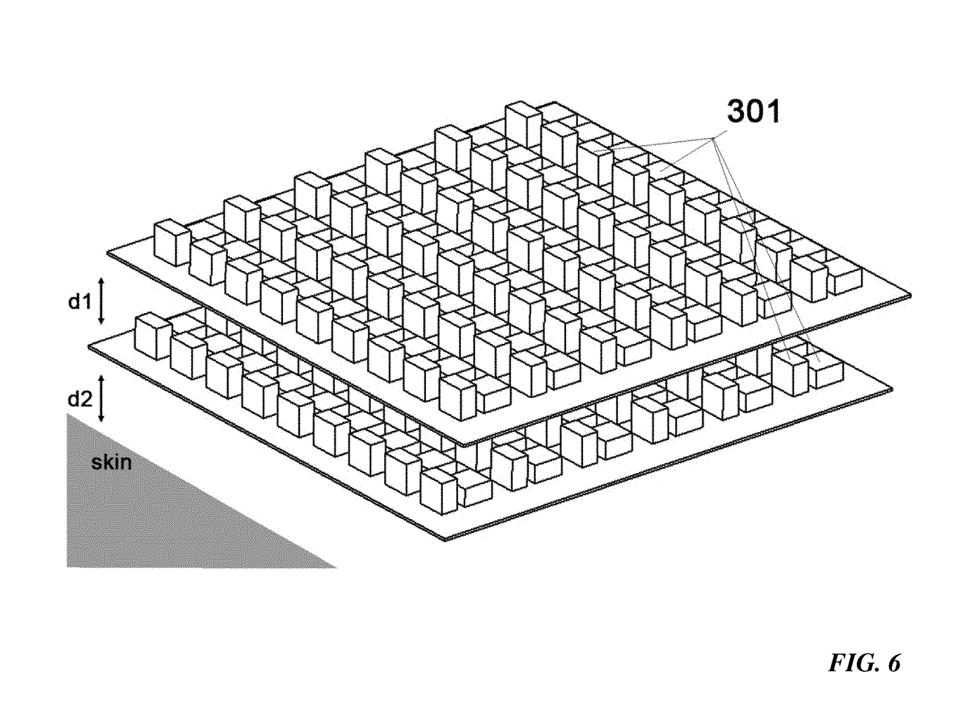

In one embodiment, the Hall Effect sensors are all oriented such that the Hall Effect sensing element is perpendicular to the ROI (FIG. 3). In another embodiment, such array of Hall Effect sensing element can be intermingled with the array of photodetectors and light emitters 204, as shown in FIG. 4. In yet another embodiment, the Hall Effect sensors in the array are oriented such that the Hall Effect sensors are positioned at any angle the ROI (FIG. 5) (e.g. 90°, 180° or any other fixed angle with respect to the ROI), or are stacked in two or more layers, at a different distances with respect to each other (e.g. d2 and d2+d1 above the skin) (FIG. 6). By altering the orientation and spatial arrangement of these magnet field detecting sensors, it is possible to accurately assess the spatial (x, y), depth (z) and rotational (φ) location of a miniaturized implant 102.

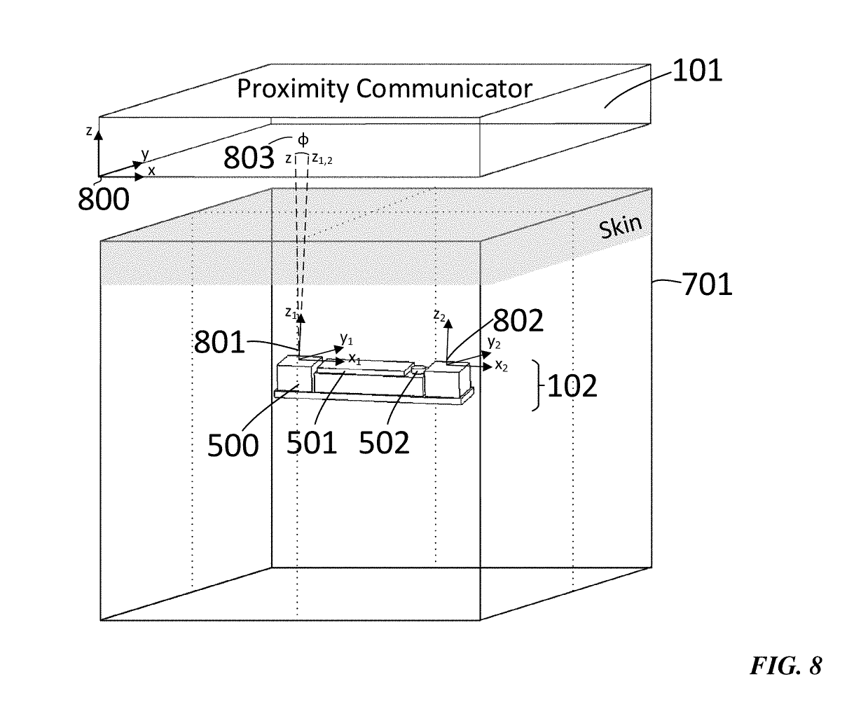

Additional circuitry 202 such as an embedded processing unit 200 or circuitry to connect to an external computer may be implemented into the proximity communicator. Software or computer algorithms are then used to store and analyze the electrical signals of the magnetic field detecting sensors. In one embodiment, the magnetic field detecting sensors produce a digital signal and an extensive array of such sensors covering a ROI can be used to represent the spatial location of the fully implantable biosensor. In a second embodiment, the analog output voltage from each hall-effect sensor over a specific surface area can be used to map the location of any magnetic material under the skin. In this embodiment, the x-y position can be determined by the array of magnetic field detecting sensors and the z-position can be determined by the analog signal strength (e.g. output voltage). Moreover, magnetic field detecting sensors can detect the orientation and rotational (φ) location of a miniaturized implant 102, i.e. the analog output voltage can be positive for north-pole facing magnets and negative for south-pole facing magnets (FIG. 8).

The proximity communicator has a second layer comprised of either a single electromagnetic radiation (EMR) source 302 and a single photodetector 303 or an array of EMR sources and photodetectors 204. The array of magnetic detection sensors 203, array of EMR sources and photodetectors 204 can be combined into a single unit. In one embodiment, the arrays are combined on multiple stacked platforms 304 (FIG. 3). In a second embodiment, the arrays of EMR sources and photodetectors 204 are combined with the array of magnetic field detecting sensors 203 on a single platform with individual component arrays embedded within each other component 400 (FIG. 4).

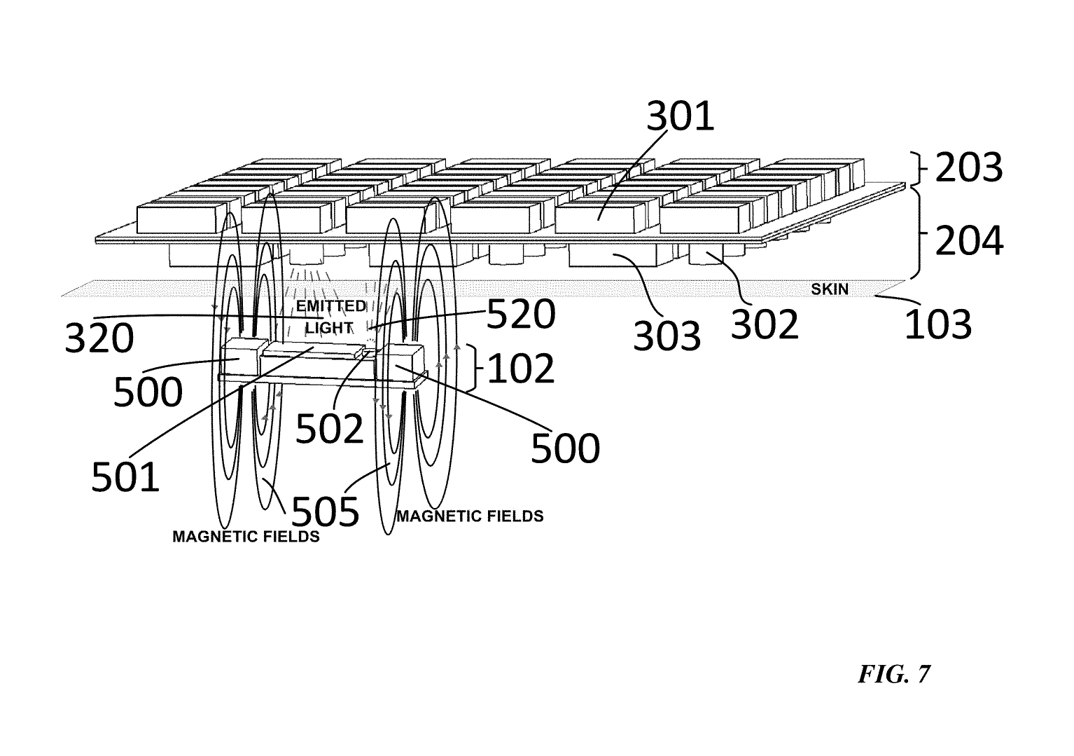

FIG. 7 illustrates how the spatial assessment of the implantable biosensor is assessed by the array of magnetic field detecting sensors 203 and in turn used to establish a line-of-sight powering and communication with the implant. One or more EMR sources 302 in the 204 array of the proximity communicator is used to provide energy to the implantable biosensor. EMR emitted from a EMR source 302 is directed toward a photovoltaic cell 501 located on the implantable biosensor. The photovoltaic cell then converts the EMR into energy that can be used to power electrical components in the biosensor. EMR can be directed toward the photovoltaic cell in multiple ways. One approach is to determine the spatial location of the biosensor, determine the orientation of the biosensor and activate one or more EMR sources 302 in the vicinity of the photovoltaic cell 501 to power the fully implantable biosensor. As shown in FIG. 7, emitted light from the external device is used to supply energy to the fully implantable biosensor. As the external device may be battery operated, the external device is capable of supplying a finite amount of energy. For continuous operations over long periods of time (e.g. weeks to months), energy consumption must be managed. This device provides a means for energy management. As one example to reduce power consumption, a limited number (e.g. one or two) of EMR sources 302 on the proximity communicator can be activated at one time.

Utilizing the magnetic materials (e.g. permanent magnets, electromagnets or micro/nanosized magnetic particles) localized within or around the implantable biosensor platform constitutes Example A. The spatial localization of such implanted biosensor platform is shown in FIG. 8. The implant 102 is equipped with one or more miniaturized permanent magnets 500, which in the case of FIG. 7, two of such magnets are located in either ends of the implant 102. These two magnets are generating a distinct magnetic field 505. This magnetic field can be readily sensed by the proximal magnetic field detecting sensor array 203, located on the external device 101. The signal from the magnetic field detecting sensor array, with the help of the appropriate circuitry 202, data acquisition 201 and micro-processor 200 analysis, can provide sufficient mapping with respect to the spatial location of the fully implantable biosensor in the ROI 701 (FIG. 8). Such spatial location analysis can take place in a millisecond to sub-millisecond time frame. This provides adequate resolution for loosely-bond external devices on users with active lifestyle (i.e. running). Software or computer algorithms are then used to store and analyze the electrical signals of each magnetic field detecting sensors.

In one embodiment, the analog output voltage from each Hall effect sensor over a specific surface area can be used to map the location of any magnetic material under the skin (e.g. the two permanent magnets 500 at either ends of the implant 102). In this embodiment, the x and y position can be determined by the relative amplitude of each of the magnetic field detecting sensors within the array. The z-position can be determined by the analog signal strength (e.g. output voltage). The array of magnetic field detecting sensors can also detect the orientation of each magnet (i.e. the analog output voltage can be positive for north-pole facing magnets and negative for south-pole facing magnets). The latter provides the means to assess the rotational angle (φ) 803 of the sensor with respect to the origin 800, arbitrarily set at one end of the external device (FIG. 8). The magnetic poles of the implant's magnets (with origins 801 and 802) can be positioned at any angle with respect to the long axis of the implant 102. One orientation may be to have the opposite magnetic poles of the two magnets facing towards the external device.

The magnetic materials utilized within the implanted biosensor of Example A might pose certain risks for elderly and/or high-risk users, who may wish to undergo magnetic resonance imaging (MRI) without the need to remove the implanted biosensor. For this, two more exemplary configurations are presented (Example B and C), which are compatible with MRI.

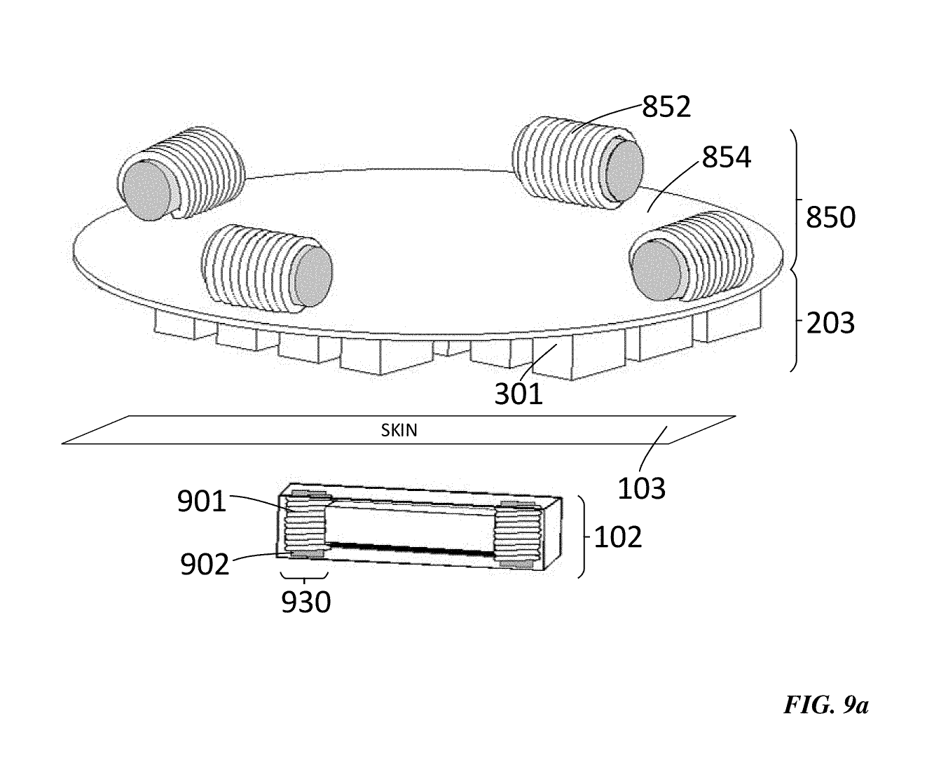

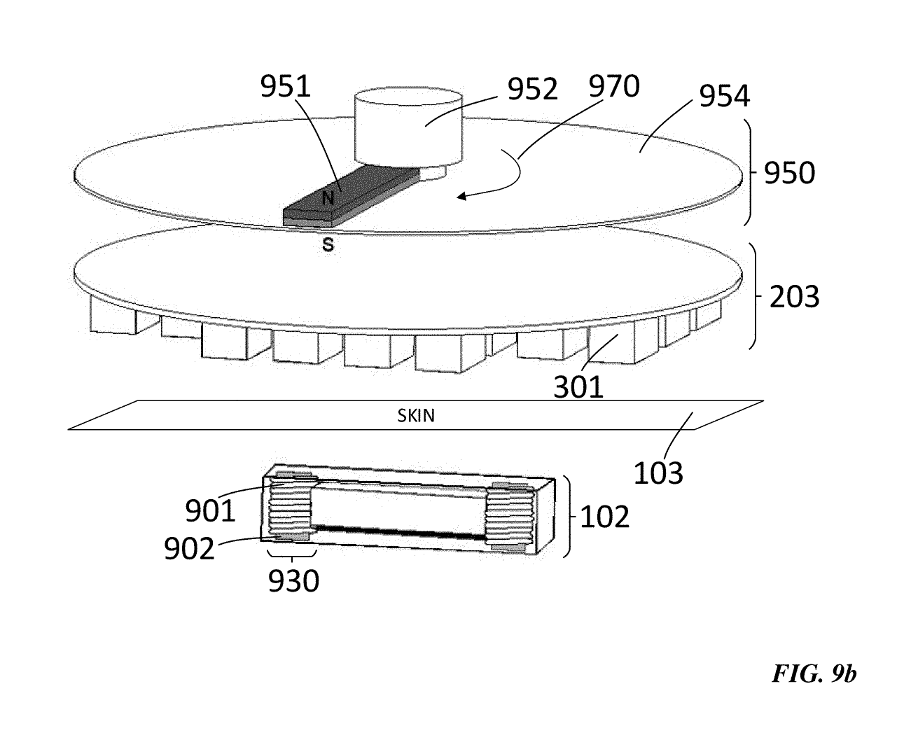

Example B utilizes magnetic interacting/polarizing materials and devices (i.e. coils) within the implanted biosensor to alter the magnetic field pattern produced by a permanent (FIG. 9a) or oscillating (FIG. 9b) magnetic field generators situated within the external device. Such magnetic field alteration is detected by the array of magnetic field detecting sensors described above and used to assess the spatial (x, y), depth (z) and rotational (φ) position of the miniaturized implant within a highly scattering tissue.

Two exemplary devices and methods for the spatial localization of the implanted biosensor using magnetic interacting/polarizing materials and devices are shown in FIG. 9. Here the implant is outfitted with magnetically interacting/polarizable materials and devices 930 (i.e. coils 901 and complex 2D and 3D architectures with or without cores 902 of magnetic polarizable substances, like spin-glass). Subcategories of magnetically polarizable material include traditional metals (Au, Pt, Pd, Cu, Al, etc.), organic conductors, graphitic materials (such as nanotubes, graphene etc.). These magnetically interacting polarizable materials and devices 930, when exposed to an external magnetic field, they can impart sufficient interaction with the external magnetic fields to slightly alter it. Static 850 and oscillating 950 magnetic fields can be used to generate an external magnetic field via permanent magnets 951 or electromagnets 852 (FIG. 9). Oscillating magnetic fields impart significantly higher interaction with magnetically polarizable materials and devices 930 as opposed to static magnetic fields. In addition, a rotating 970 magnetic field 950 facilitates the individual magnetic field sensors 301 of the 203 array to periodically de-saturate from the strong magnetic field of the proximal permanent magnets or electromagnets (FIG. 9b). This will facilitate optimal operation of the entire magnetic field detecting sensor array. Along these lines, the electromagnets 852 placed on a surface 854 can be sequentially powered to emulate a rotating magnetic field (FIG. 9a). Spatial mapping and position determination of the implantable sensor is facilitated by contrasting the response of the magnetic field sensing array 203 in the presence and absence of the implant. The magnetic field sensing array 203 response in the absence of the implant is obtained and stored in memory from a site without an implant.

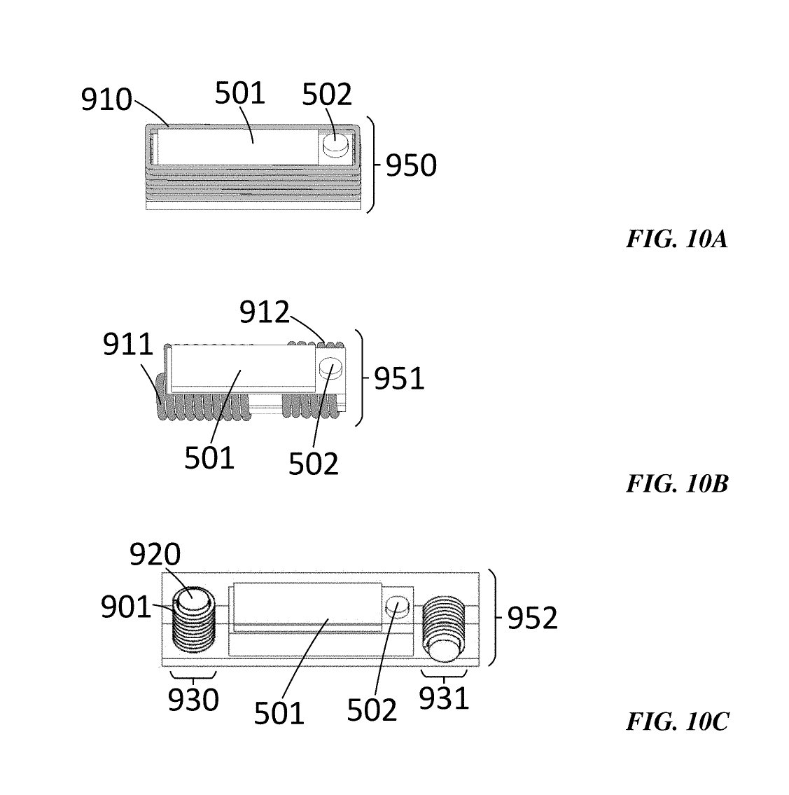

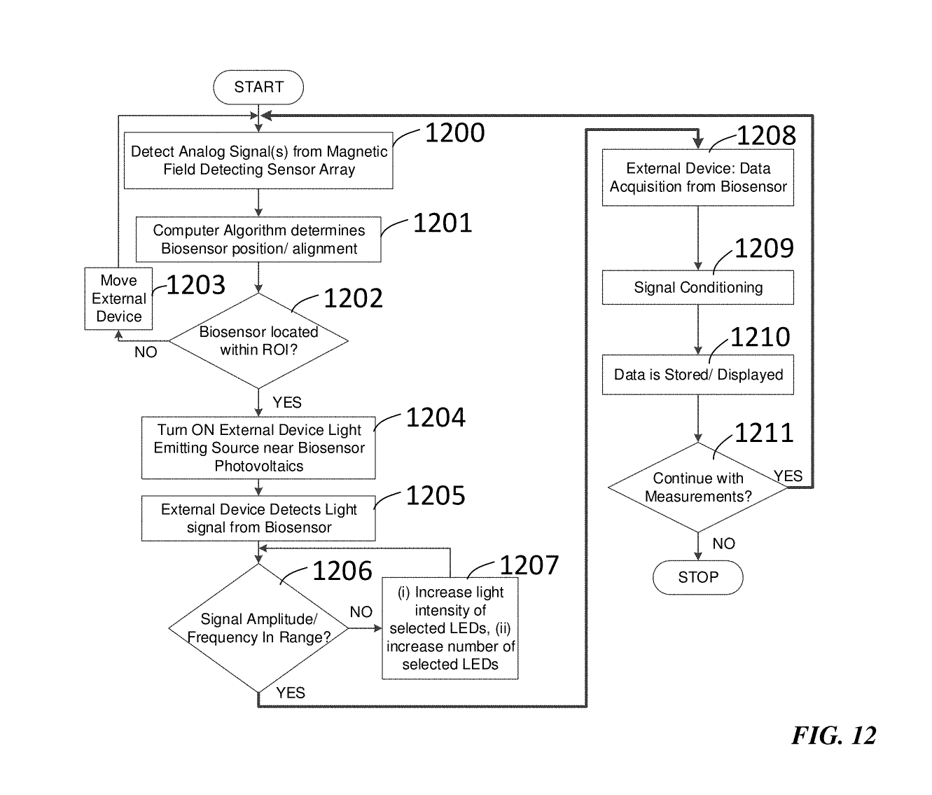

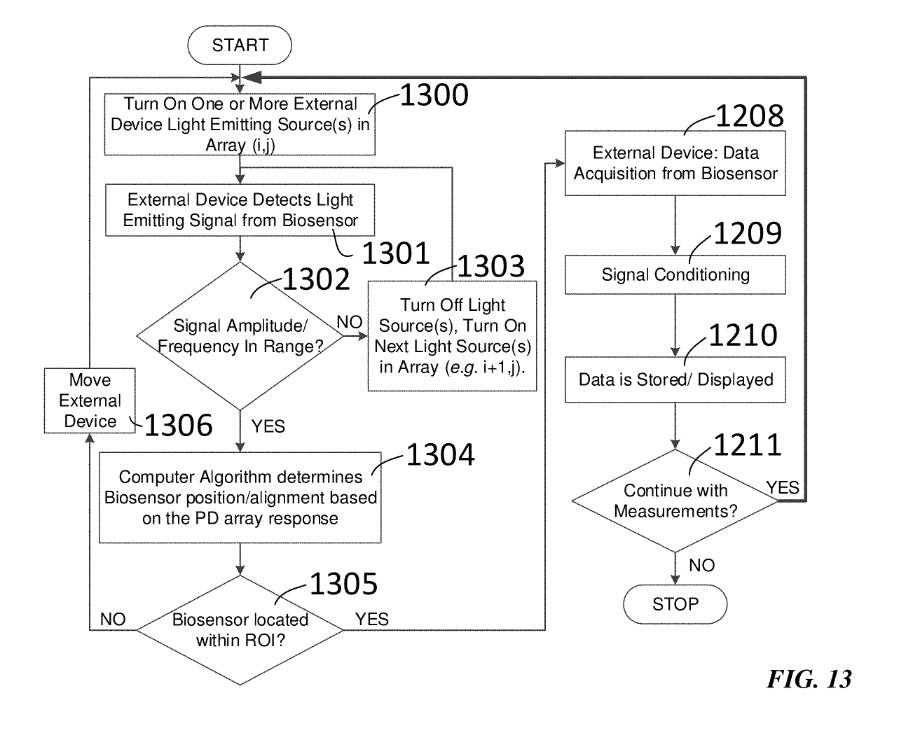

FIG. 10 provides exemplary configurations of magnetically interacting/polarizable materials (i.e. coils) within or on the implanted biosensor. FIG. 10A is composed of a single coil 910 wrapped around the outside of the biosensor. FIG. 10B shows two coils at different sizes wrapped within the biosensor. FIG. 12C consists of miniature electromagnetics 931 placed within the implant. Here, close and open-loop coils (i.e. 910, 911, 912, and 901) of different length and filling (with and without magnetic polarizable cores 920) are depicted. The three exemplary architectures of FIG. 10 are suitable for spatial detection (x, y), depth (z) and rotational (φ) position of the miniaturized implant (i.e. 950, 951 and 952) within highly scattering tissue.

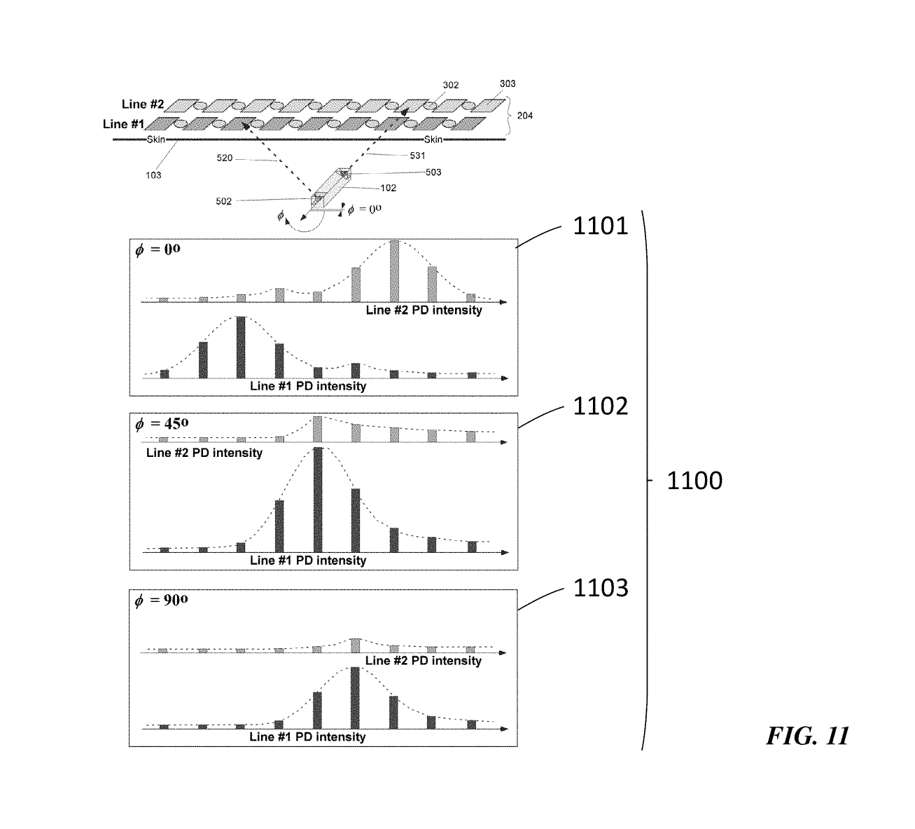

Example C describes another exemplary device and method for the spatial localization of the implant without the use of permanent magnets that can be incompatible with MRI. This approach negates completely the need for the array of magnetic field detecting sensors 203 and relies solely on the array of photodetector (PD) and LEDs 204 of the external device (proximity communicator) to map the emission from the two on-board LEDs or lasers (502 and 503) within the implantable biosensor 102 (FIG. 11). The two on-board light sources are oriented at 90° with each other in order to provide differential PD response upon φ rotation (although their relative orientation can greatly vary). FIG. 11 illustrates three exemplary PD line responses for φ of 0°, 45° and 90°. Since the front on-board light source 502 lines up with PD line #1 and the back on-board light source 503 lines up with PD line #2, different response patterns will be obtained depending on the specific rotation of the implant. These patterns can be stored in the memory of microprocessor 200 and used to analyze the observed response to decipher the rotational (φ) angle of the miniaturized implant within a highly scattering tissue. The depth (z) can be assessed by the separation maxima between Line #1 and Line #2 of PDs (larger separation means greater depth). The density of the photodetector array (i.e. number of PD sensors per area), implant depth, and light scattering power of the skin that the implant is located, affect the mapping resolution of the PD array 204. Such resolution can be ultimately reduced down to 25 microns

### Description of Method: Determine Biosensor Spatial Location and Alignment

One exemplary method to determine which light emitter(s) 302 is powered by the external device is based on a computer algorithm structure outlined in FIG. 12. The magnetic field detecting sensor array in the proximity communicator 203 converts the magnetic field produced by the biosensor magnets 500 into an analog electrical signal 1200. A computer algorithm then determines the spatial location of the biosensor and the alignment of the biosensor 1201. The algorithm establishes if the biosensor is located within a region of interest (ROI) 1202. As an example, the ROI 701 is a geometrically defined zone located under the proximity communicator in the vicinity of the light emitting/photodetector array 204 and magnetic field detecting sensor array 203 of the proximity communicator. A yes/no-decision is performed, whether the biosensor implant is located within the ROI 1202. In the case that the biosensor implant is not located in the ROI, the algorithm requests the user or subject to move 1203 the proximity communicator and the process is repeated from the beginning.

In the case that the biosensor implant is in the ROI, one or more light emitting sources 302 located in the vicinity of the biosensors photovoltaic cell(s) 501 turns ON 1204. Upon activation, electricity is generated by the photovoltaic cell(s) 501 and the implantable biosensor sends a signal via its on-board light emitting source 502 to the external device 1205. A yes/no-decision is performed by the external device to determine if signal characteristics (e.g. amplitude and frequency) produced by the biosensor are within a pre-determined range of values 1206. Upon the values being outside of the pre-determined range, then the algorithm instructs it from the following options 1207: (i) increase the power of the selected light emitting source(s); and (ii) increase the number of selected light emitting sources in the vicinity of the biosensor 1207.

In addition, the signal amplitude/frequency In Range comparison 1206 accommodates biosensor rotation and tilt by activating the light emitting source(s) at locations that would provide higher intensity light at an angle with respect to the rotated biosensor, if necessary. Upon the values being at or within the pre-determined range, the external device acquires the data from the biosensor 1208, performs signal processing 1209, and stores/displays the data 1210. A yes/no-decision is performed to either continue with the measurements or stop 1211. Upon a continuation, the entire process is repeated at the initial stage. This method provides sufficient power management and facilitates continuous operation of the biosensor even upon large movements (e.g. up to ±2.5 cm) of the watch-like, external device (or other type of external device).

A second exemplary method to determine the spatial location of the biosensor can be accomplished by using the array 204 of light emitting sources (herein defined as i,j array of LEDs where individual LEDs in the array are identified as LEDij) and photodetectors (herein defined as i,j array of PD where individual PDs in the array are identified as PDij) in the external device. In the example described below, the biosensor has one or more light emitting source at known angles with respect to the biosensor. Upon initiation, a computer algorithm either activates one or more light emitting sources in the external device light emitting source array 1300. An array of photodiodes is time-division multiplexed to determine if the biosensor is emitting a signal. In this manner, the emitted light from the biosensor is analyzed at each photodetector in the external device 1301. At each photodiode, the amplitude and frequency of the signal is compared to be within a specified range 1302. Upon the emitted signal amplitude or frequency being out of the specified range, the light emitting source or set of sources (e.g. i, j) is deactivated and another the light emitting source or set of sources (e,g. i+1, j) is activated 1303. Upon the emitted signal amplitude or frequency being within the specified range, the computer algorithm collects the input signals from the time-division multiplexed photodetectors and determines the biosensor position and alignment 1304. Such information can provide either a two-dimensional (x,y) or three-dimensional (x,y,z) mapping of the implant. The biosensor location is then determined to be within the region of interest (ROI) 1305.

Another method to determine the spatial location of the implant is to turn on all the LEDs in the LEDij array and sequentially interrogate each of PDij output to identify the spatial x-y position of the implant. Upon the sensor not being within the ROI, the above process repeats and the user is instructed to physical move the proximity communicator to a new location 1305. Upon the biosensor being within the ROI, the external device acquires the data from the biosensor 1208, performs signal processing 1209, and stores/displays the data 1210. A yes/no-decision is performed to either continue with the measurements or stop 1211. The exemplary methods stated above are not exhaustive and only two examples of methods that can be used to determine the spatial location/alignment of the implanted biosensor while establishing optical communication between the biosensor and the external device.

### Improved Patient Compliance

The proximity communicator described hereto provides a means to increase patient compliance with respect to wearing the proximity communicator. The proximity communicator is intended to provide for minimal discomfort as the device can be loosely affixed to the subject's body. Moreover, the automatic biosensor alignment and communicator protocols provide a means for the subject to move the device and still obtain accurate and reproducible data. For example, in one embodiment the proximity communicator can be affixed to a wrist of a human subject and normal daily routines that involve movements of the wrist would not interfere with the communicator to and from the biosensor.

It should be appreciated that while the invention has been described with reference to an exemplary embodiment, it will be understood by those skilled in the art that various changes, omissions and/or additions may be made and equivalents may be substituted for elements thereof without departing from the spirit and scope of the invention. Moreover, embodiments and/or elements of embodiments disclosed herein may be combined as desired. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims and/or information. Moreover, unless specifically stated any use of the terms first, second, etc. do not denote any order or importance, but rather the terms first, second, etc. are used to distinguish one element from another.

What is claimed is:

1. A wearable system for the spatial detection a fully implantable miniaturized biosensor with in a body tissue, using minimal energy, the system comprising;
an external control unit,
a miniaturized, fully implantable biosensor platform,
wherein said external control unit comprises of an array of magnetic field detecting sensors, an array of light emitters, and an array of light photodetectors,
wherein said external control unit also contains a microprocessor which interfaces with powering source, data acquisition module, display, magnetic field sources, and other components,
wherein said miniaturized biosensor platform is outfitted with light powered photovoltaic cells and one or more light emitters to optical transmit the detected concentration values of various analytes,
wherein said miniaturized biosensor platform comprises of one or more miniaturized magnets,
wherein the magnetic field of said miniaturized magnets is sensed and imaged by the said magnetic field detecting sensor array in the external control unit to provide the assessment of the spatial (x, y) position, depth (z) and rotational (φ) state of the implantable biosensor platform,
wherein said spatial (x, y) position allows to turn on one or more light emitters in the said array of the external control unit, that are in a line-of-sight alignment with the miniaturized biosensor platform,
wherein said depth and rotational coordinates information is used by the microprocessor in the external control unit to adjust the output light intensity of the selected light emitters, as well as power adjacent light emitters to compensate for the rotation of the said photovoltaic cells,
wherein said spatial and rotational position is used by the microprocessor to turn on one or more photodetectors in the said array of the external control unit that are also aligned with the miniaturized biosensor platform.
wherein said changes in the spatial position and orientation of the external control unit with respect to the miniaturized biosensor platform is assessed to account for random motion caused by intense physical activity of the user.

2. The device of claim 1 wherein the said assessment of the location of a miniaturized implantable biosensor within a body tissue is between 1 microsecond and 1000 milliseconds range.

3. The device of claim 1 wherein the said assessment of the location of a miniaturized implantable biosensor within a body tissue is between 10 microns and 10 millimeters range.

4. The device of claim 1 wherein the said miniaturized magnets is comprised of high strength magnetic material selected from a list samarium, iron, ferrite, samarium boron garnet.

5. The device of claim 1 wherein the said magnetic field detecting sensors array is composed of multiple Hall effect sensors and giant magnetoresistance sensors.

6. The device of claim 5 wherein half of the said magnetic field detecting sensors are oriented parallel and half are oriented perpendicular with respect to their resting substrate

7. The device of claim 5 wherein the said magnetic field detecting sensors array is distributed within two layers separated by a distance that varies from 0.1 to 10 mm.

8. The device of claim 1 wherein the said miniaturized magnets within the implantable biosensor platform is replaced with one or more miniaturized electromagnets.

9. The device of claim 8 wherein the said miniaturized electromagnets within the implantable biosensor platform are electrically activated to generate a magnetic field around the implant.

10. The device of claim 1 wherein the said miniaturized magnets on the biosensor platform is replaced with one or more magnetically susceptible coils that distort the magnetic field generated be the said magnetic field sources residing within the external control unit.

11. The device of claim 10 wherein the said magnetic field is either static or oscillating.

12. The device of claim 11 wherein the said oscillating magnetic field is generated by a rotating magnet that resides within the external control unit.

13. The device of claim 11 wherein the said oscillating magnetic field is sequentially activating electromagnets residing within the external control unit.

14. A method for spatial detection of a miniaturized fully implantable biosensor within a body tissue that comprises magnetic alignment and minimizes energy usage via an algorithm facilitating alignment for both optical powering and optical communication units,
wherein said algorithm is located in the microprocessor of an external control unit which interfaces with a miniaturized biosensor platform,
wherein said algorithm interfaces with an array of magnetic field detecting sensors, an array of light emitters, and an array of light photodetectors within the said external control unit,
wherein said algorithm also interfaces with powering source, data acquisition module, display, magnetic field sources, and other components within the said external control unit,
wherein said algorithm interfaces with the said miniaturized biosensor platform through its light powered photovoltaic cells and one or more light emitters that optically transmits the detected concentration values of various analytes to the said external control unit,
wherein said algorithm senses the position of the miniaturized biosensor platform through the mapping of the magnetic field generated by one or more miniaturized magnets located on it, and imaged by the said magnetic field detecting sensor array in the external unit to provide the precise assessment of the spatial (x, y) position, depth (z) and rotational (φ) state of the implantable biosensor platform,
wherein said algorithm uses the precise spatial (x, y) position to turn on one or more light emitters in the said array of the external control unit, which are aligned by line-of-sight with the miniaturized biosensor platform,
wherein said algorithm uses the depth and rotational coordinates information to adjust the output light intensity of the selected light emitters, as well as power adjacent light emitters to compensate for the rotation of the said photovoltaic cells
wherein said algorithm uses the precise spatial and rotational position to turn on one or more photodetectors in the said array of the external control unit that are also aligned with the miniaturized biosensor platform.
wherein said algorithm accounts for changes in the spatial position and orientation of the external control unit with respect to the miniaturized biosensor platform to account for random motion caused by intense physical activity of the user.

15. The method of claim 14 wherein the said assessment of the location of a miniaturized implantable biosensor within a body tissue is between 1 microsecond and 1000 milliseconds range.

16. The method of claim 14 wherein the said assessment of the location of a miniaturized implantable biosensor within a body tissue is between a 10-micron and 10-millimeter range.

17. The method of claim 14 wherein by orienting half of the said magnetic field detecting sensors of the array perpendicular to the other half, depth and rotational accuracy of the implanted biosensor platform is improved.

18. The method of claim 14 wherein the dividing the said magnetic field detecting sensors array into two layers separated by a distance that varies from 0.1 to 10 mm, depth and rotational accuracy of the implanted biosensor platform is improved.

19. The method of claim 14 wherein the said miniaturized magnets within the implantable biosensor platform is replaced with one or more miniaturized electromagnets in order to render the implant allowable to undergo MRI imaging.

20. The method of claim 14 wherein the said miniaturized magnets on the biosensor platform is replaced with one or more magnetically susceptible coils in order to render the implant allowable to undergo MRI imaging.

21. A method for spatial detection of a miniaturized fully implantable biosensor within a body tissue that comprises optical alignment and minimizes energy usage via an algorithm facilitating alignment for both optical powering and optical communication units,
wherein said algorithm is located in the microprocessor of an external control unit which interfaces with a miniaturized biosensor platform,
wherein said algorithm interfaces with an array of light emitters, and a array of light photodetectors within the said external control unit,
wherein said algorithm also interfaces with powering source, data acquisition module, display, and other components within the said external control unit,
wherein said algorithm interfaces with the said miniaturized biosensor platform through its light powered photovoltaic cells and a pair of light emitters oriented at 90° from each other and at 45° with respect to the bottom of the said external control unit,
wherein said algorithm senses the position of the miniaturized biosensor platform through the mapping of the intensity generated on the array of light photodetectors to provide the precise assessment of the spatial (x, y) position, depth (z) and rotational (φ) state of the implantable biosensor platform,
wherein said algorithm uses the precise spatial (x, y) position to turn on one or more light emitters in the said array of the external control unit, which are aligned by line-of-sight with the miniaturized biosensor platform,
wherein said algorithm uses the depth and rotational coordinates information to adjust the output light intensity of the selected light emitters, as well as power adjacent light emitters to compensate for the rotation of the said photovoltaic cells
wherein said algorithm uses the precise spatial and rotational position to turn on one or more photodetectors in the said array of the external control unit that are also aligned with the miniaturized biosensor platform.
wherein said algorithm accounts for changes in the spatial position and orientation of the external control unit with respect to the miniaturized biosensor platform to account for random motion caused by intense physical activity of the user.

22. The method of claim 21 wherein the said assessment of the location of a miniaturized implantable biosensor within a body tissue is between 1 microsecond and 1000 milliseconds range.

23. The method of claim 21 wherein the said assessment of the location of a miniaturized implantable biosensor within a body tissue is between 10 microns and 10 millimeters range.

24. The method of claim 21 wherein the said pair of light emitters on the miniaturized implant are oriented at an angle that varies from 0° to 180° and their alignment from the said bottom of the external control unit varies from 0° to 180°.

25. The method of claim 21 wherein the said algorithm first powers the entire array of light emitters at the external control unit to activate emission from the said pair of light emitters on the miniaturized implant.

26. The method of claim 21 wherein the said algorithm stores the intensity response generated on the array of light photodetectors in the absence of a miniaturized implant and uses it as a frame of reference for comparing the mapping of the said intensity generated on the array of light photodetectors to provide the precise assessment of the spatial (x, y) position, depth (z) and rotational (φ) state of the implantable biosensor platform.
